(19) United States
(12) Patent Application Publication
(10) Pub. No.: US 2011/0020480 A1
(43) Pub. Date: Jan. 27, 2011

(54) USE OF BENZOXAZINOIDS-CONTAINING CEREAL GRAIN PRODUCTS FOR HEALTH-IMPROVING PURPOSES

(75) Inventors: Inge Sindbjerg Fomsgaard, Roskilde (DK); Anne Garfield Mortensen, Tappernoje (DK); Preben Bach Holm, Valby (DK); Per Langkjaar Gregersen, Roskilde (DK)

Correspondence Address: THE NATH LAW GROUP, 112 South West Street, Alexandria, VA, 22314, US

(73) Assignee: AARHUS UNIVERSITET, Arhus C (DK)

(21) Appl. No.: 12/933,195

(22) PCT Filed: Mar. 19, 2009
(86) PCT No.: PCT/DK2009/050064

§ 371 Date: Sep. 17, 2010

(30) Foreign Application Priority Data
Mar. 19, 2008 (EP) 08005140.2

(51) Int. Cl. A61K 36/899; A61P 35/00; A61P 31/04; A61P 29/00; A61P 25/00; A61P 25/04

(52) U.S. Cl. 424/750

(57) ABSTRACT

The invention relates to the use of benzoxazinoids-containing grains of cereals for the production of food and beverage products with health-improving effects. Especially the invention relates to the use of benzoxazinoids-containing grains of cereals for the production of bread baked on rye grains, wheat, spelt, rye and/or spelt sprouts for health-improving purposes. The health-improving effects according to the invention are obtained through the CNS stimulating effects of said benzoxazinoid-containing food and beverage products. These effects are appetite suppression, enhancement of mood, improved sexual function, relieve of fibromyalgia and sleep apnea disorders. Other CNS-related disorders are included as well. The health-improving effects of the invented food products containing benzoxazinoids also include anti-cancer activity, anti-inflammatory effects, analgesic effects and antibacterial effects. The total content of benzoxazinoids was maintained in the bread through the baking process and it can be calculated that the daily amount of rye bread needed to obtain a CNS-stimulating effect is about 1.5-240 g.

### TECHNICAL FIELD

The invention relates to the use of mature grains of benzoxazinoids-containing cereals for the production of food and beverage products with health-improving effects.

The invention further relates to hydrothermically processed mature grains of cereals and uses thereof for the production of food and beverages for health-improving purposes.

In one aspect the invention relates to the use of benzoxazinoid-containing grains of cereals for the production of food—such as bread, muesli, flakes—and beverages made with whole or homogenized grains of cereals such as rye, maize, wheat, spelt, kamut, einkorn, other Triticum species, barley and oat. The invention also relates to food—such as bread, muesli, flakes—and beverages made of hydrothermically processed cereal grains.

### BACKGROUND

Benzoxazinoids are secondary metabolites that hitherto have been found in green plant parts of monocotyledoneous plants including cereal plants. They have been studied extensively during the last decade due to their properties as defence compounds in agricultural crops. The chemical structure of benzoxazinoids resembles the structure of the signalling compounds melatonin, serotonin and tryptophan. Several benzoxazinoids are patented due to their CNS effects, as potential drugs for use as antidepressants, aphrodisiacs and appetite suppression or sexual stimulators (U.S. Pat. No. 6,667,308, United States Patent Application 2006/0160795, WO/2006/017281).

Benzoxazinoids have been shown to have anti-inflammatory and analgesic effects and some have been shown to inhibit growth of human breast cancer cell lines and prostate cell lines. According to a number of studies performed in rodents, the most studied benzoxazinoid 6-methoxy-benzoxazolin-2-one (6-MBOA) acts in the pineal-hypothalamus-pituitary axis, possibly as a melatonin agonist and at the α- and β-adrenergic receptors in its own right. A study of 6-MBOA on human males showed a significant positive effect on depression or mood.

An estimated 10% of the population in the industrialised countries will suffer from major depressive episodes during their lifetime and many more will experience lesser bouts. Clinical depression can be very debilitating and even less severe depressions cause lost productivity and relationship problems and the societal costs are very high.

In a recent summary published in the European Journal of Neurology it is stated that brain disorders (psychiatric, neurological and neurosurgical diseases together) figure amongst the leading causes of disease and disability. Moreover, World Health Organization (WHO) data suggest that brain disorders cause 35% of the burden of all diseases in Europe. In the same summary, it is estimated that 127 million Europeans out of a population of 466 million currently live with a brain disorder, estimating the total annual cost of brain disorders in Europe to be EUR 386 billion in 2004. Of these, mental disorders constituted 62% of the total cost, making these diseases responsible for great socio-economic consequences. The numbers greatly emphasize the importance of developing new strategies in treating these disorders.

It is well-established that young seedlings and sprouts of cereals such as wheat and spelt, grown in the field or in agar plates, contain significant amounts of benzoxazinoids. WO 20067017281 A describes that certain benzoxazinoids, derived from green plant parts of monocotyledonous plants e.g. cereal plants, may be used for inducing calming effects and anti anxiety in humans. WO 2006/017281 A summarizes that the compound exist in the earlier growth phases of monocotyledonous plants. WO 2006/017281 A disclose that benzoxazinoids are obtainable from monocotyledonous plants by “specific harvesting and drying conditions” and that the compounds of the invention are obtained from plants via circumstances differing from the usual manner in which the plants are handled for the terminal product.

FR-A 2 865 899 does not mention benzoxazinoids or other secondary metabolites in cereal products. FR-A-2 865 899 discloses a dietetic bread used for stabilizing the blood sugar level of overweight people and thus obtaining a feeling of satiety. At the same time this invented bread has a property of rehydration in the stomach, which gives a feeling of “filled stomach” and thus also provides a feeling of satiety. In contrast, the present invention relates to the effect of benzoxazinoids in bread on humans, which is a reduction of the desire to eat, obtained through the stimulation of the central nervous system.

FR-A-2 840 772 discloses that flour made of cereal sprouts adds a better taste and a better appearance to the bread without loosing the technological qualities of traditional flour.

US 2006/0264429, U.S. Pat. No. 6,667,308 and US 2006/0223796 disclose specific benzoxazinoids as pharmaceuticals for the treatment of obesity, depression, sexual dysfunction, fibromyalgia, sleep apnea, diabetes, hyperglycemia and other CNS-related disorders. These patents cover the use of the pure compounds as medicinal products as well as extracts of these compounds obtained from monocotyledonous plants at their early growth stages.

It is of great innovative value and of high societal relevance, if the consumption of food products prepared from grains of cereals, such as bread made from grains of rye, wheat and spelt varieties, may alleviate the symptoms of depression, resulting in great social benefits. Accordingly, there is in the art a need for cereal products, e.g. cereal-containing food products and beverages including functional food products and nutraceuticals, bread products and bread with a well-defined content of health-improving benzoxazinoids.

However, until recently, it was believed that mature cereal grains (also called kernels), as well as disintegrated grains (e.g. flour) made from such cereal grains, did not contain benzoxazinoids. A number of earlier publications have underlined that benzoxazinoids are formed in the early growth stages of the plants and said publications did not find benzoxazinoids in mature cereal grains.

### SUMMARY OF THE INVENTION

During the experiments leading to the present invention, it was surprisingly found that this belief was not true. The discovery of the presence of benzoxazinoids in mature cereal grains (the usual terminal product of cereal crops) was done recently in the lab of the inventors. Without wishing to be bound by theory, the inventors believe that the content of significant amounts of benzoxazinoids in grains of cereals has not previously been observed, as the compounds may not have been liberated from the grains for measurement in previous investigations. However, the experiments leading to the present invention has shown that the compounds are in fact present in significant amounts in the grains, as it was observed that food products prepared form the grains according to the invention contained a much higher content of benzoxazinoids than was to be expected from investigation of the unprepared grains.

Further it was surprisingly observed that simple treatment with water of the grains prior to the preparation of food products resulted in an even higher content of benzoxazinoids in the final product.

The inventors of the present invention have identified and quantified the benzoxazinoid content and profile in mature grains from different varieties of rye, wheat and grains from spelt, kamut, einkorn and in bread products produced there from, and found that the benzoxazinoids are present in the grains and further that the levels of benzoxazinoids are maintained and even elevated through the baking process.

Accordingly, in a first aspect the invention relates to the use of grains or disintegrated grains of benzoxazinoid-containing cereals for the manufacturing of a food product for a human or animal subject to improve the health of the subject. The invention also relates to a method of improving the health of a human or animal subject by ingesting grains or disintegrated grains of benzoxazinoid-containing cereals. The health-improving effects according to the invention are selected among CNS stimulating effects, anti-cancer activity, anti-inflammatory effects, analgesic effects and antibacterial effect.

Preferably, the benzoxazinoids-contents in the cereal grain products are their naturally present content.

In a second aspect the invention relates to a method of producing an increased content of benzoxazinoids in grains of benzoxazinoid-containing cereals, the method comprising the steps of a) contacting the grains with water, and b) drying the grains, and c) optionally repeating the steps a) and b), during a period of at least two days. Preferably this treatment is followed by a step d) of heat treating the grains or, optionally, disintegrated grains at a temperature of above 60° C. Thereby an increased content of benzoxazinoids has been observed to occur.

In a third aspect the invention relates to food product comprising grains or disintegrated grains of benzoxazinoid-containing cereals characterised in that the cereal grains have been pre-treated by being contacted with water and dried one or more times during a period of at least two days.

### DEFINITIONS

Throughout this disclosure, various terms generally understood by persons skilled in the art are used. However, several terms are used with specific meanings and are meant as defined by the following.

The term “cereal” is meant to comprise plants from the grass family of which the starchy grains are used as food and plants from other plant families (some times named pseudo-cereals) that produce starchy grains which are used as food.

The term “grain” is meant to comprise whole grains of cereal crops. Preferably the grains comprise the germ, bran and the endosperm and are thus defined as whole grains. Preferably the grains are “mature” meaning that they have been harvested at a normal maturation state. In one aspect, however, the term grain may comprise over-matured grains meaning that they have been harvested at a later stage than the normal maturation state.

By the term “disintegrated grain” is means grains that are cut, grinded or milled into smaller fragments. Disintegrated grains include flour. Preferably, however, the flour should be made from whole grains and sieving should be omitted.

By the phrase “substantially maintained through the baking process” is meant that at least 50%, preferably >75%, more preferably >90%, more preferably >95%, most preferably up to 100% of the benzoxazinoids are maintained through the baking process.

The term “benzoxazinoid” is meant to comprise any lactam, hydroxamic acid, benzoxazolinone, methyl-derivate, their glycosidic derivatives and other derivatives with the same basic structure. Preferably the benzoxazinoids are one or more of DIBOA, DIBOA-Glc, DIMBOA, DIMBOA-Glc, DIM₂BOA, DIM₂BOA-Glc, BOA, MBOA, M₂BOA, HBOA, HBOA-Glc, HMBOA, HMBOA-Glc, HM₂BOA, HM₂BOA-Glc, DHBOA, DHBOA-Glc, 4-O-Me-DIBOA, 4-O-Me-DIBOA-Glc, HDMBOA, HDMBOA-Glc including any isomers, steroisomers and enantiomers thereof. Included are also chlorinated and further hydroxylated derivatives of the above mentioned compounds. The benzoxazinoids of the present invention are grouped in four families of which basic structures and examples are defined in Table 1.

TABLE 1
Benzoxazinoids grouped into families based on basic structures
Family 1
Hydroxamic acids

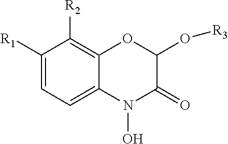

| R₁ | R₂ | R₃ | Acronym |
|---|---|---|---|
| R₁ = H | R₂ = H | R₃ = H | DIBOA |
| R₁ = H | R₂ = H | R₃ = Glc | DIBOA-Glc |
| R₁ = OCH₃ | R₂ = H | R₃ = H | DIMBOA |
| R₁ = OCH₃ | R₂ = H | R₃ = Glc | DIMBOA-Glc |
| R₁ = OCH₃ | R₂ = OCH₃ | R₃ = H | DIM₂BOA |
| R₁ = OCH₃ | R₂ = OCH₃ | R₃ = Glc | DIM₂BOA-Glc |

Family 2
Benzoaxazolinones

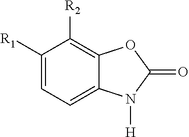

| R₁ | R₂ | Acronym |
|---|---|---|
| R₁ = H | R₂ = H | BOA |
| R₁ = OCH₃ | R₂ = H | MBOA |
| R₁ = OCH₃ | R₂ = OCH₃ | M₂BOA |

Family 3
Lactams

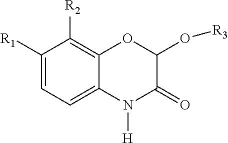

| R₁ | R₂ | R₃ | Acronym |
|---|---|---|---|
| R₁ = H | R₂ = H | R₃ = H | HBOA |
| R₁ = H | R₂ = H | R₃ = Glc | HBOA-Glc |
| R₁ = OCH₃ | R₂ = H | R₃ = H | HMBOA |
| R₁ = OCH₃ | R₂ = H | R₃ = Glc | HMBOA-Glc |
| R₁ = OCH₃ | R₂ = OCH₃ | R₃ = H | HM₂BOA |
| R₁ = OCH₃ | R₂ = OCH₃ | R₃ = Glc | HM₂BOA-Glc |
| R₁ = OH | R₂ = H | R₃ = H | DHBOA |
| R₁ = OH | R₂ = H | R₃ = Glc | DHBOA-Glc |

Family 4
Methyl drivatives

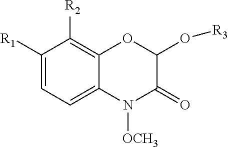

| R₁ | R₂ | R₃ | Acronym |
|---|---|---|---|
| R₁ = H | R₂ = H | R₃ = H | 4-O-Me-DIBOA |
| R₁ = H | R₂ = H | R₃ = Glc | 4-O-Me-DIBOA-glc |
| R₁ = OCH₃ | R₂ = H | R₃ = H | HDMBOA |
| R₁ = OCH₃ | R₂ = H | R₃ = Glc | HDMBOA-Glc |

Table 2 defines the structure, systematic names, acronyms, formula and molecular mass of the listed specific compounds and isomers hereof (with the hydroxy- or meth-oxy-group in another position either in the benzene ring or in the heterocyclic ring) and other derivates hereof that maintain the same basic structure, including derivatives with one or more glycosidic molecule attached and including halogenated derivates.

TABLE 2
Benzoxazinoids compounds with structure, systematic names, acronyms, formula and molecular mass

| Systematic name | Acronym | Mass | Formula |
|---|---|---|---|
| 2,4-dihydroxy-(2H)-1,4- benzoxazin-3(4H)-one | DIBOA | 181 | 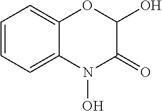 |
| 2-O-β-D- glucopyranosyloxy- 4-hydroxy-(2H)-1,4- benzoxazin-3(4H)-one | DIBOA-Glc | 343 | 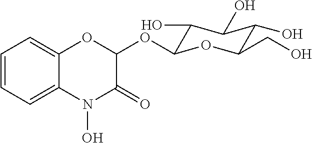 |
| 2,4-dihydroxy-7- methoxy-(2H)-1,4- benzoxazin-3(4H)-one | DIMBOA | 211 | 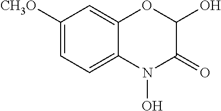 |
| 2-O-β-D- glucopyranosyloxy-4- hydroxy-7-(2H)- methoxy-1,4- benzoxazin-3(4H)-one | DIMBOA-Glc | 373 | 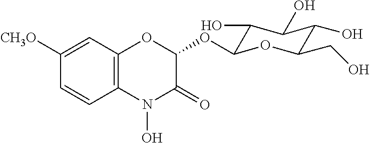 |
| 2,4-dihydroxy-7,8- dimethoxy-(2H)-1,4- benzoxazin-3(4H)-one | DIM₂BOA | 241 | 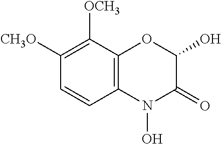 |
| 2-O-β-D- glucopyranosyloxy-4- hydroxy-7,8-dimethoxy- (2H)-1,4-benzoxazin- 3(4H)-one | DIM₂BOA-Glc | 403 | 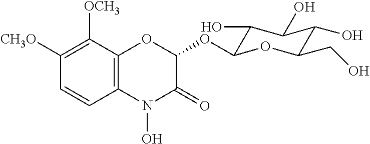 |
| 2-(2H)-hydroxy-1,4- benzoxazin-3(4H)-one | HBOA | 165 | 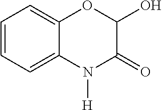 |
| 2-O-β-D- glucopyranosyloxy-1,4- benzoxazin-3(4H)-one | HBOA-Glc | 327 | 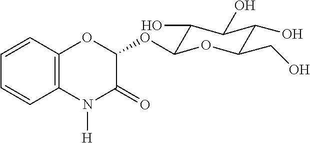 |
| 2-hydroxy-7-methoxy- (2H)-1,4-benzoxazin- 3(4H)-one | HMBOA | 195 | 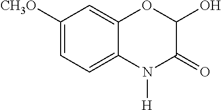 |
| 2-O-β-D- glucopyranosyloxy-7- methoxy-(2H)-1,4- benzoxazin-3(4H)-one | HMBOA-Glc | 357 | 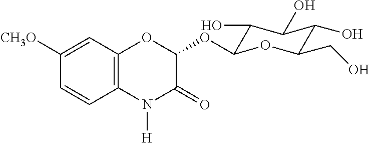 |
| 2-hydroxy-7,8- dimethoxy-(2H)-1,4- benzoxazin-3(4H)-one | HM₂BOA | 225 | 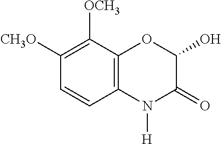 |
| 2-O-β-D- glucopyranosyloxy-7,8- dimethoxy-(2H)-1,4- benzoxazin-3(4H)-one | HM₂BOA-Glc | 387 | 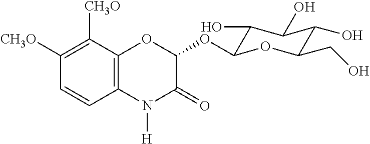 |
| 2-hydroxy-4,7- dimethoxy-(2H)- 1,4-benzoxazin-3(4H)- one | HDMBOA | 225 | 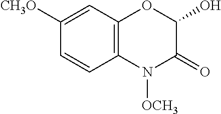 |
| 2-O-β-D- glucopyranosyloxy-4,7- dimethoxy-(2H)-1,4- benzoxazin-3(4H)-one | HDMBOA-Glc | 387 | 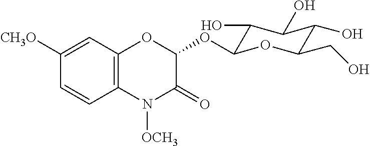 |
| benzoxazolin-2-one | BOA | 135 | 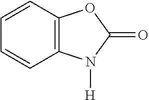 |
| 6-methoxy-benzoxazolin- 2-one | MBOA | 165 | 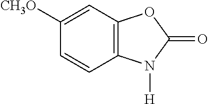 |
| 6,7-dimethoxy- benzoxazolin-2-one | M₂BOA | 195 | 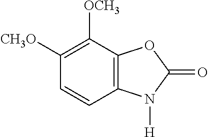 |

### DETAILED DESCRIPTION OF THE INVENTION

The inventors' identification and quantification of the benzoxazinoid content and profile in grains of different strains of rye, wheat and spelt and in their bread products, and the finding that the benzoxazinoids are maintained through the baking process, has made it possible to develop bread with a suitable benzoxazinoid profile and content in relation to the desired health-improving purpose(s).

Thus our results show for the first time the presence of benzoxazinoids in bread baked from grains of cereals. Especially high content was found in baked rye bread. However, bread baked from 4 wheat varieties and from the cereal strains kamut, einkorn and spelt also contained significant levels of benzoxazinoids, which has never been shown before. Especially bread baked of the wheat variety Robigus and of spelt contained benzoxazinoids at a concentration level that resembles the two rye varieties.

Thus, in a first aspect the invention relates to the use of grains or disintegrated grains of benzoxazinoid-containing cereals for the manufacturing of food products for a human or animal subject to improve the health of the subject. The invention also relates to a method of improving the health of a human or animal subject by ingesting grains or disintegrated grains of benzoxazinoid-containing cereals. If using disintegrated grains, a higher content of benzoxazinoids was observed if whole grains, i.e. all parts of the grains including the germ, the bran and the endosperm was used. Accordingly, in a preferred aspect of the invention disintegrated whole grains are used without sieving

The benzoxazinoids according to the invention are lactams, hydroxamic acids, benzoxazolinones, methyl-derivates, their glycosidic derivatives and other derivatives with the same basic structure. Preferably, the benzoxazinoids are one or more of DIBOA, DIBOA-Glc, DIMBOA, DIMBOA-Glc, DIM₂BOA, DIM₂BOA-Glc, BOA, MBOA, M₂BOA, HBOA, HBOA-Glc, HMBOA, HMBOA-Glc, HM₂BOA, HM₂BOA-Glc, DHBOA, DHBOA-Glc, 4-O-Me-DIBOA, 4-O-Me-DIBOA-Glc, HDMBOA, HDMBOA-Glc including any isomers, steroisomers and enantiomers thereof. Included are also chlorinated and further hydroxylated derivatives of the above mentioned compounds.

In one aspect the cereal grains are grains of rye. In a further aspect the cereal grains are grains of maize. In a further aspect the cereal grains are grains of wheat. In a further aspect the cereal grains are grains of kamut. In a further aspect the cereal grains are grains of einkorn. In a further aspect the cereal grains are grains of spelt. In a further aspect the cereal grains are grains of wild barley. In a further aspect the cereal grains are grains of rice. In a further aspect the cereal grains are grains of oat. In a further aspect the cereal grains are grains of sorghum. In a further aspect the cereal grains are grains of millet. In a further aspect the cereal grains are grains of teff. In a further aspect the cereal grains are grains of buckwheat. In a further aspect the cereal grains are grains of amaranth. In a further aspect the cereal grains are grains of quinoa. In a further aspect the cereal grains are grains of durum. In a further aspect the cereal grains are grains of triticale. In a further aspect the cereal grains are derived from cross-breedings of the above mentioned cereals.

The food products according to the invention are cereal-grain-containing food products and beverages, e.g. functional food products and/or nutraceutical, and bread products, e.g. baked bread, e.g. baked with grains from one or more of wheat, spelt, rye, maize, einkorn, kamut where the benzoxazinoids-content in the cereal product is substantially maintained through the baking process.

The health-improving effects according to the invention are obtained through the CNS stimulating effects of said benzoxazinoid-containing food and beverage products. These effects are appetite suppression, enhancement of mood, improved sexual function, relieve of fibromyalgia and sleep apnea disorders. Other CNS-related disorders are included as well. The health-improving effects of the invented food products containing benzoxazinoids also include anti-cancer activity, anti-inflammatory effects, analgesic effects and antibacterial effects. Thus, in one aspect of the invention the health-improving effect is appetite suppression by CNS stimulation. In one aspect of the invention the health-improving effect is enhancement of mood by CNS stimulation. In one aspect of the invention the health-improving effect is improved sexual function by CNS stimulation. In one aspect of the invention the health-improving effect is relieve of fibromyalgia by CNS stimulation. In one aspect of the invention the health-improving effect is relieve of sleep apnea disorders by CNS stimulation. In one aspect of the invention the health-improving effect is anti-cancer activity. In one aspect of the invention the health-improving effect are anti-inflammatory effects. In one aspect of the invention the health-improving effects are analgesic effects. In one aspect of the invention the health-improving effect is antibacterial effect.

In a further aspect the invention relates to the use as defined above, where the animal is a mammal, e.g. a human.

The therapeutic dose of 6-MBOA for treating depressive states of mind is approx. 15 mg daily and the cumulative daily dose for obtaining appetite suppression in overweight women was 15 mcg (U.S. Pat. No. 6,667,308). Assuming that all the analyzed benzoxazinoids have a molar pharmacological activity similar to 6-MBOA, and if the content of benzoxazinoids in rye bread (Example 1 Table 4, this application) is converted to molar equivalents of 6-MBOA, these doses can be obtained by eating in the range of 1.45 g-242 g of a ryebread purchased in the Supermarket Irma and in the range of 0.9-155 g of a ryebread, baked in our laboratory of the rye variety Picasso.

WO 2006/017281 A discloses that the therapeutic dose varied between 5 mcg and 60 mg. If the activity of the analyzed benzoxazinoids are assumed to be similar to the activity of 6-MBOA, measured in mcg, the dose of 60 mg could be obtained by eating around 300 g of a bread, baked of svedjerug or by eating 20 g Picasso grains processed according to the invention.

Surprisingly, the inventors found that a simple pretreatment of the grains resulted in an increased content of benzoxazinods. This simple pre-treatment was the contacting of the grains with water (e.g. by placing of the grains in a perforated tray), followed by a resting period (e.g. in the tray) and/or a subsequent drying period. The duration of the pre-treatment should be at least two days, and more preferably at least three days and most preferably four days. It was observed that treatment wherein the grains were humidified with water and allowed to rest at a temperature of 22° C. for a period of at least 2 days, such as between 2-6 days or even more preferred between 2-5 days such as 2 days or 3 days or 4 days, resulted in an increased content of benzoxazinoid.

Preferably, the grains were contacted with water once a day. More preferably, the grains should be in contact with running water several times a day for at least 2 days without being immersed in water for longer periods of time exceeding 1 hour.

Preferably, grains after contacting with water should be maintained or rest or dry at a temperature of between 5° C. and 35° C.

Optionally, grains can be soaked in water for less than 1 minute once or twice a day and subsequently left for resting and/or drying (e.g. in a perforated tray).

During the pre-treatment period according to this aspect of the invention the grains were humidified with running water at least 2 times. Preferably the grains are humidifled with water at least once a day. Even more preferably, the grains are humidified with water 2 times a day. It is not preferred that the grains are soaked in water for longer periods of time as it was surprisingly shown that continuous contact with water resulted in a decreased content of benzoxazinoids in the final grain containing food product. Accordingly, in a preferred aspect the contact with running water has a duration of between 1 second and 1 hour. Even more preferred the contact with water has a duration of less than 30 minutes. Even more preferred the contact with water has a duration of less than 20 minutes. Even more preferred the contact with water has a duration of less than 1 minute.

Preferably the amount of running water used in the contact with grains is more than 20 ml and less than 1 liter per 15 grams of grain. Even more preferred the amount of running water used for contacting the grains is between 200 and 400 ml in the first wetting, while even more preferred the amount of water used in the subsequent wettings is between 100 and 200 ml water.

Thus, the invention also relates to a method of producing an increased content of benzoxazinoids in grains of benzoxazinoid-containing cereals, the method comprising the steps of a) contacting the grains with water, and b) resting and/or drying the grains, and c) optionally repeating the steps a) and b), during a period of at least two days.

Optionally grains can be kept between 0 and 5° C. in the resting period and optionally the treatment period can thus be extended to more than 4 days.

Pre-treatment according to this aspect of the invention results in the formation of hydrothermically processed grains with a high content of benzoxazinoids in several cases. When hydrothermically processed grains of the variety kamut were dried and grinded and a bread was baked in which this flour was used as part of the ingredients, benzoxazinoids were still present in the baked bread and the concentration of benzoxazinoids was higher than in a bread, baked of fine and coarse ground Kamut flour. Bread baked of the hydrothermically processed grains had a very attractive smell and taste, as expressed by the laboratory personnel.

Without wishing to be bound by theory, the inventors believe that both chemical and enzymatic processes take place during the described hydrothermic processing of grains and also during the subsequent preparation of for instance bread. The inventors also believe that our hydrothermic process firstly provokes the enzymatic processes to start, when pouring water over the grains and secondly the resting period without soaking in water resembles a kind of drought stress that increases the formation of the compound substantially.

It was observed that the benzoxazinoids-content in the cereal product is substantially maintained through the heating (baking) process. Thus, in a further aspect the food product according to the invention is a product which has been heated. Heated products include bread product(s), e.g. baked bread, e.g. baked with rye grains and/or grains from one or more of cereal grains. The determined content of benzoxazinoids is increasing through the processing of the grains. The determined concentration in bread>concentration in flour>concentration in grains. The reason fort his observed effect is believed to be that the benzoxazinoids to varying extents are in insoluble-bound forms which are thought to be librated by grinding, milling and even baking of the grains.

Thus in one preferred aspect the grains are disintegrated prior to processing into a food product. In another aspect the grains are heat treated prior to or in the process of processing into a food product. Preferably, the heat treatment occur at a temperature of between 60° C. and 300° C., more preferred at between 70° C. and 220° C. In a preferred aspect the grains are baked at temperatures of between 150° C. and 200° C. in the process of processing into a food product.

In a preferred aspect the food product is baked bread.

### EXAMPLES

### Materials and Methods

### Extraction of Benzoxazinoids

Freeze-dried samples of rye grains were crushed and homogenised with a Waring blender before extraction by an Accelerated Solvent Extraction 200 system (Dionex)(ASE). Five grams of glowed chemically inert Ottawa sand (particle size 20-30 mesh, Fisher Chemicals) was added to the 33 ml extraction cells. Subsequently, 0.1 g of the freeze-dried and homogenised sample was transferred to the extraction cell and a filter was placed on top of the sample. Thereafter, the extraction cell was filled with glowed glass balls. The eluent was 80% methanol, 19% water, and 1% glacial acetic acid (v/v). The protocol for the ASE extraction was the following: preheat for 5 min, heat for 5 min, static for 3 min, flush 80%, purge for 50 s, 4 cycles, pressure 107 Pa, and temperature 80° C. Extracts were collected in vials, which were filled with eluent to maintain 44 g weight for all extracts, and stored at −20° C. until chemical analysis. Unless stated otherwise, the extraction method above was used through the examples.

### Example 1

### Chemical Analysis of Benzoxazinoids

The extracts were filtered on a Sartorius SRP 15 0.45 μm filter (PTFE membrane) and diluted with water in a 1:1 ratio. An Applied Biosystems MDS Sciex API 2000 liquid chromatography-triple quadrupole mass spectrometer (LCMSMS) with turbo electrospray ionisation in a positive multiple reaction monitoring (MRM) mode was used for the chemical analysis. The chromatographic separation was performed at a flow rate of 0.2 ml/min at 30° C. with an injection volume of 20 μl. The column was a Hypersil BDS C18 (2.1×250 mm, 5 μm). The A-eluent contained 10% methanol and 90% filtered milliQ water (v/v) with 20 mM glacial acetic acid. The B-eluent was methanol containing 20 mM glacial acetic acid. The gradient contained the following: 90% A for 1 min followed by a linear gradient to 30% A for 8 min and isocratic elution for the following 7 min, and subsequently a 1 min ramp back to 90% A and reequilibration for 7 min. The total run time of the analysis was 23 min. The first 8 min were run to waste.

Standard solutions of pure reference compounds were used for identification of the benzoxazinoids in the rye grain samples based on a comparison of fragmentation patterns and retention times. The standard curves were applied to a quadratic function with a weighting of 1/x since there were more data points at the lower part of the curve (correlation coefficient >0.99).

The purpose of this example was to determine the benzoxazinoids content in different cereals and bread products, especially baked whole rye bread. The cereals or rye bread were freeze-dried, the benzoxazinoids extracted and the analysis performed on HBOA, HMBOA, BOA, MBOA, DIBOA-Glc, DIMBOA-Glc, DIBOA, DIMBOA, HMBOA-Glc and HDMBOA-Glc, as described above. The results for the benzoxazinoids are shown in Table 3.

TABLE 3
Benzoxazinoids content in different cereal sources

| Benzoxazinoids | HBOA | BOA | DIBOA-Glc | HMBOA-Glc |
|---|---|---|---|---|
| source | (mcg/g) | (mcg/g) | (mcg/g) | (mcg/g) |
| Rye bread A | 4.4 | 15.6 | 106.8 | 35.4 |
| Rye bread B | 5.1 | 15.3 | 129.2 | 35.4 |
| Spelt flake A | n.d | n.d | 9.6 | 7.7 |
| Spelt flake B | n.d | n.d | 9.3 | 7.0 |
| Wheat Astron A | n.d | n.d | 1.3 | 0.9 |
| Wheat Astron B | n.d | n.d | 1.3 | 0.6 |
| Wheat Ritmo A | n.d | 1.0 | 1.8 | 0.7 |
| Wheat Ritmo B | n.d | n.d | 1.7 | 1.0 |

The total (quantifiable) amount of benzoxazinoids was calculated by summing up the individual amounts of the in Table 4 mentioned benzoxazinoids. It was assumed that 1) the health-improving daily doses of benzoxazinoids are in a range of 90 μg to 15 mg MBOA, respectively; 2) the health-improving effect(s) of each of the in Table 4 mentioned benzoxazinoids are similar to the effect of the corresponding amount of MBOA; and 3) the water content of the rye bread is 60%. Based on these assumptions, the amount of rye bread containing 90 μg and 15 mg MBOA, respectively, was calculated (see Table 4). The dose range assumption is built on the experiments by Rosenfeld et al (2006), who estimated a cumulative daily dosis of 90 μg in a weight loss experiment and a daily dosis of 15 mg in an antidepressant study.

TABLE 4
Amount of rye bread corresponding to a content of MBOA equivalents at 90 mcg and 15 mg

| | | | | | | Amount (g) of rye bread correspond- | | | |
|---|---|---|---|---|---|---|---|---|---|
| | | | | | | ding to 90 mcg and 15 mg MBOA, re- | | | |
| | mcg benzoxazinoids/ | | | | | spectively (g dw in parentheses) | | | |
| Benzoxazinoid | g dry weight | | Mw mcg/ | micromole | | “Irma” | | “Picasso” | |
| Rye bread | “Irma” | “Picasso” | micromole | “Irma” | “Picasso” | 90 mcg | 15 mg | 90 mcg | 15 mg |
| HMBOA-Glc | 35 | n.d. | 358 | 0.10 | | | | | |
| HBOA | 5 | 1.6 | 165 | 0.03 | 0.010 | | | | |
| BOA | 16 | 36 | 135 | 0.12 | 0.26 | | | | |
| DIBOA-Glc | 130 | 220 | 343 | 0.38 | 0.64 | | | | |
| MBOA | n.d. | 0.1 | 165 | | 0.001 | | | | |
| DIMBOA-Glc | n.d. | 0.5 | 373 | | 0.001 | | | | |
| DIBOA | n.d. | 2.3 | 181 | | 0.013 | | | | |
| HBOA-Glc | n.d. | 12 | 327 | | 0.04 | | | | |
| isomer of | n.d. | 4.8 | 343 | | 0.014 | | | | |
| 6-OH-HBOA-Glc | | | | | | | | | |
| total | 186 | 278 | | 0.63 | 0.98 | 1.45 | 242 | 0.93 | 155 |
| | | | | | | (0.87) | (145) | (0.56) | (93) |

| | | | |
|---|---|---|---|
| Comparative | Amount, mg | | Amount, micromole |
| MBOA | 0.09 | 165 | 0.55 |
| | 15 | 165 | 90.9 |

(mcg are microgram)

### Example 2

### Benzoxazinoid Profile and Content in Grains and Flour Before, and in Bread after, Baking

### Baking of Rye Bread

The “Picasso” rye bread was manufactured as follows: 300 ml water, ¼ table spoon salt, 175 g cracked Picasso rye grains, 175 g wheat flour, 175 g rye flour and 2 tea spoons dry yeast were added to a Melissa baking apparatus 643-043 and mixed and baked in 3 h 40 min. at program 3.

The “Irma” whole meal rye bread was purchased from Irma Denmark. Both kinds of rye bread were analysed as described below.

Method for analysis of benzoxazinoids in rye grains before and after baking chemical analysis.

The extracts were filtered on a Sartorius SRP 15 0.45 mcgm filter (PTFE membrane) and diluted with water at a 1:1 ratio. An Applied Biosystems MDS Sciex API 3200 liquid chromatography-Ion Trap quadrupole mass spectrometer (LCMSMS) with turbo electrospray ionisation in a negative multiple reaction monitoring (MRM) mode was used for the chemical analysis. The chromatographic separation was performed at a flow rate of 0.2 mL/min at 30° C. with an injection volume of 20 mcgL. The column was a Synergi 4u Polar (2.0×250 mm,). The A-eluent contained 7% acetoni-trile and 93% filtered milliQ water (v/v) with 20 mM glacial acetic acid. The B-eluent was 78% acetonitrile containing 20 mM glacial acetic acid. The gradient was as follows: 0-1 min 84% A, 1-5 min 82% A, 5-22 min 70% A, 22-30 min 0% A, 30-35 min 0% A, 35-39 min 84% A and re-equilibration for 9 min. The total run time of the analysis was 48 min.

The pure reference compounds were used for identification of the compounds based on a comparison of fragmentation pattern and retention times. The standard curves were applied to a quadratic function with a weighting of 1/x since there were more data points at the lower part of the curve (correlation coefficient >0.99).

TABLE 5
Benzoxazinoids content in different cereal sources before and after baking

| | | | | | | | isomer of | |
|---|---|---|---|---|---|---|---|---|
| Benzoxazinoids | HBOA | MBOA | DIMBOA-Glc | DIBOA | BOA | HBOA-Glc | 6-OH-HBOA-Glc* | DIBOA-Glc |
| source | (mcg/g) | (mcg/g) | (mcg/g) | (mcg/g) | (mcg/g) | (mcg/g) | (mcg/g) | (mcg/g) |
| Flour - | n.d. | 0.10 | 0.36 | 0.72 | 1.3 | 3.9 | 26.0 | 250.3 |
| Picasso | | | | | | | | |
| Flour - | 0.28 | 0.17 | 1.03 | 1.21 | 3.1 | 4.3 | 27.3 | 219.9 |
| Visello | | | | | | | | |
| Flour - | n.d. | n.d. | n.d. | n.d. | n.d. | 0.4 | 23.5 | 7.5 |
| Agronom | | | | | | | | |
| Flour - | 0.16 | 0.04 | 0.33 | 0.76 | 0.6 | 1.4 | 25.3 | 61.1 |
| Rotari | | | | | | | | |
| Grains - | n.d. | n.d. | 0.31 | 0.67 | 1.4 | 2.8 | 21.8 | 185.7 |
| Picasso | | | | | | | | |
| Grains - | 0.25 | 0.15 | 0.85 | 1.29 | 2.6 | 3.2 | 23.4 | 171.3 |
| Visello | | | | | | | | |
| Grains - | n.d. | n.d. | 0.18 | n.d. | 0.2 | 0.3 | 20.7 | 6.1 |
| Agronom | | | | | | | | |
| Grains - | n.d. | 0.04 | 0.31 | 0.54 | 0.5 | 1.0 | 20.9 | 39.6 |
| Rotari | | | | | | | | |
| Ryebread - | 1.62 | 0.11 | 0.53 | 2.28 | 35.7 | 10.8 | 4.7 | 220.1 |
| Picasso | | | | | | | | |
| Ryebread - | 0.82 | 0.35 | 1.30 | 0.81 | 17.7 | 15.3 | 8.5 | 325.3 |
| Visello | | | | | | | | |
| Ryebread - | 0.52 | 0.17 | n.d. | | 10.2 | 2.6 | 5.9 | 41.5 |
| Agronom | | | | | | | | |
| Ryebread - | 0.97 | 0.26 | 0.67 | 1.10 | 20.7 | 6.8 | 7.6 | 114.6 |
| Rotari | | | | | | | | |

*quantified on basis of HBOA-Glc standard

### Example 3

Additional baking trials were performed using the same procedure as described in Example 2. The content of benzoxazinoids were quantified in the ingredients and in the final baked product using an extraction procedure similar to the procedure that was used in the Example 1 and 2 and using an LCMSMS analytic procedure similar to the one used in Example 2.

The method for preparation of flour was as follows: Coarse flour was prepared by treating 500 g grains in a Retsch mortar for 2 minutes at 50 g. Fine flour was produced by grinding in a Fidibus 21 grinder. None of the flours were sieved.

Baking trials were performed with the following varieties/species: Svedjerug (an old Nordic rye variety with a high protein content), Picasso (a rye variety commonly grown in Denmark), Kamut, Ambition (Danish winter wheat variety), Smugler (Danish winter wheat variety), Ritmo (Danish winter wheat variety) Robigus (Danish winter wheat variety), Einkorn (old Triticum species) and Spelt (old Triticum species).

The recipe for all the breads was as follows:

300 ml water, 1 teaspoon salt, 175 g “blank” wheat flour (fine flour purchased in the supermarket), 175 g finely ground flour of the selected variety, 175 g coarsely ground flour of the selected variety, 2 teaspoon dry yeast. The bread was baked in a Melissa baking machine 643-043 in program 3.

Table 6 shows that DIBOA-glc was the dominating compound in all varieties and it was extractable and detectable in both fine and coarse flour of all varieties except coarse flour of spelt. The extraction performed better in finely ground flour than in coarse ground flour in many cases, —however the differences were not of great importance. Surprisingly, the concentration of extractable concentrations increased substantially in baked bread when compared to the theoretical calculation, done on basis of the benzoxazinoid content of the ingredients.

Breads baked of the two rye varieties (svedjerug and Picasso) resulted with the highest concentration of benzoxazinoids.

To assure that the addition of blank wheat flour did not disturb the conclusions, a bread was also made of pure blank wheat flour. This bread only contained 4.1 μg/g DIBOA-glc; 0.4 μg/g DIBOA and 0.5 μg/g HBOA-glc and no detectable concentrations of the other compounds. In all cases in Table 6, the blank wheat flour thus only had minor influence on the concentrations found in the trials.

### Example 4

Production of hydrothermically processed grains was done in trays from Bergs Bio salad. 15 g of grains were distributed evenly in a perforated tray size 14×14 cm. Water was gently poured over the grains twice a day during 4 days. Each time water was poured over the grains, this had a duration of ca 20 seconds. During this process the water passed freely through the perforated tray and the grains were never left soaking in water. The first washing was done with 300 ml water and the following with 150 ml water. After 4 days, the hydrothermically proccessed grains were harvested from the tray and dried in an oven at 50° C. for 6 hours.

The dried hydrothermicdally processed grains were homogenized and the benzoxazinoid concentration was determined using the similar extraction and chromatographic method as in Example 3.

Hydrothermic processing of cereal grains with this technique resulted in significant increases in extractable benzoxazinoid content (Table 6), compared to the finely or coarsely ground grains.

### Example 5

Bread was also baked with flour prepared of dried sprouts of Kamut. The recipe was as follows:

300 ml water, 1 teaspoon salt, 175 g “blank” wheat flour (fine flour purchased in the supermarket), 175 g fine Kamut flour, 175 g ground Kamut sprout flour, 2 teaspoon dry yeast. The bread was baked in a Melissa baking machine 643-043 in program 3.

Table 7 shows the content of benzoxazinoids in this bread compared to bread baked as in Example 3:

300 ml water, 1 teaspoon salt, 175 g “blank” wheat flour (fine flour purchased in the supermarket), 175 g fine Kamut flour, 175 g coarse Kamut flour, 2 teaspoon dry yeast. The bread was baked in a Melissa baking machine 643-043 in program 3.

The benzoxazinoid concentrations are seen in Table 7.

TABLE 6
Benzoxazinoid concentrations in fine ground flour, coarse ground flour, baked bread and sprouts of cereals. Results
from this table are referred to in Example 3 and Example 4. HTP-grains are hydrothermically processed grains

| | | | | | | DiBOA- | DIM-BOA- | DI- | DIM- | HBOA- |
|---|---|---|---|---|---|---|---|---|---|---|
| | | BOA | MBOA | HBOA | HMBOA | glc | glc | BOA | BOA | glc |
| Variety/species | | mcg/g* | mcg/g* | mcg/g* | mcg/g* | mcg/g* | mcg/g* | mcg/g* | mcg/g* | mcg/g* |
| | “Blank” wheat flour | 0.0 | 0.0 | 0.0 | 0.0 | 1.7 | 0.0 | 0.0 | 0.0 | 0.0 |
| Svejderug | Finely ground flour | 1.1 | 0.0 | 0.1 | 0.0 | 105.1 | 0.0 | 12.7 | 1.5 | 2.4 |
| | Coarse ground flour | 0.0 | 0.0 | 0.0 | 0.0 | 83.7 | 0.7 | 3.1 | 2.3 | 2.4 |
| | Baked bread | 15.1 | 0.0 | 0.9 | 0.0 | 143.5 | 0.6 | 34.8 | 0.0 | 4.6 |
| | HTP-grains | 3.1 | 1.8 | 1.8 | 0.4 | 529.5 | 31.9 | 624.9 | 28.7 | 4.6 |
| Theoretical content in baked bread** | | 0.4 | 0.0 | 0.0 | 0.0 | 63.5 | 0.2 | 5.3 | 1.3 | 1.6 |
| Rye: | Finely ground flour | 2.2 | 0.0 | 0.2 | 0.0 | 288.6 | 0.5 | 3.1 | 0.0 | 4.5 |
| Picasso | Coarse ground flour | 0.8 | 0.0 | 0.0 | 0.0 | 186.8 | 0.0 | 4.6 | 0.0 | 3.2 |
| | Baked bread | 12.6 | 0.0 | 0.0 | 0.0 | 147.6 | 0.5 | 14.4 | 0.0 | 6.1 |
| | HTP-grains | 8.5 | 6.5 | 3.6 | 1.1 | 1031.4 | 81.6 | 1619.2 | 62.6 | 7.8 |
| Theoretical content in baked bread** | | 1.0 | 0.0 | 0.1 | 0.0 | 159.0 | 0.2 | 2.6 | 0.0 | 2.6 |
| Kamut | Finely ground flour | 0.0 | 0.0 | 0.0 | 0.0 | 3.5 | 0.0 | 1.6 | 0.0 | 0.0 |
| | Coarse ground flour | 0.0 | 0.0 | 0.0 | 0.0 | 1.2 | 0.0 | 0.0 | 0.0 | 0.0 |
| | Baked bread | 0.0 | 0.0 | 0.1 | 0.0 | 12.1 | 0.0 | 1.1 | 0.0 | 0.9 |
| | HTP-grains | 0.5 | 34.3 | 1.5 | 15.9 | 235.5 | 87.6 | 200.9 | 189.7 | 6.4 |
| Theoretical content in baked bread** | | 0.0 | 0.0 | 0.0 | 0.0 | 2.1 | 0.0 | 0.5 | 0.0 | 0.0 |
| Wheat: | Finely ground flour | 0.0 | 0.0 | 0.0 | 0.0 | 1.5 | 0.0 | 1.9 | 0.0 | 0.0 |
| Ambition | Coarse ground flour | 0.0 | 0.0 | 0.0 | 0.0 | 2.0 | 0.0 | 0.7 | 2.1 | 0.0 |
| | Baked bread | 0.8 | 0.0 | 0.2 | 0.0 | 28.1 | 0.0 | 1.2 | 0.0 | 1.9 |
| | HTP-grains | 0.0 | 52.0 | 0.9 | 9.7 | 126.6 | 116.3 | 64.0 | 373.5 | 2.3 |
| Theoretical content in baked bread** | | 0.0 | 0.0 | 0.0 | 0.0 | 1.8 | 0.0 | 0.8 | 0.7 | 0.0 |
| Wheat: | Finely ground flour | 0.0 | 0.0 | 0.0 | 0.0 | 2.6 | 0.0 | 1.2 | 0.0 | 0.2 |
| Smugler | Coarse ground flour | 0.0 | 0.0 | 0.0 | 0.0 | 2.4 | 0.0 | 0.6 | 0.0 | 0.0 |
| | Baked bread | 0.5 | 0.0 | 0.0 | 0.0 | 18.7 | 0.0 | 0.0 | 0.0 | 1.3 |
| | HTP-grains | 1.4 | 29.4 | 0.6 | 6.4 | 242.2 | 93.1 | 67.2 | 89.6 | 2.2 |
| Theoretical content in baked bread** | | 0.0 | 0.0 | 0.0 | 0.0 | 2.2 | 0.0 | 0.6 | 0.0 | 0.1 |
| Wheat: | Finely ground flour | 0.0 | 0.0 | 0.2 | 0.0 | 4.9 | 0.0 | 3.5 | 0.0 | 0.6 |
| Ritmo | Coarse ground flour | 0.0 | 0.0 | 0.0 | 0.0 | 3.1 | 0.0 | 1.4 | 0.0 | 0.5 |
| | Baked bread | 0.0 | 0.0 | 0.2 | 0.0 | 14.9 | 0.0 | 0.2 | 1.0 | 1.1 |
| | HTP-grains | 0.3 | 25.3 | 0.2 | 5.4 | 69.9 | 111.6 | 0.0 | 98.9 | 0.5 |
| Theoretical content in baked bread** | | 0.0 | 0.0 | 0.1 | 0.0 | 3.2 | 0.0 | 1.6 | 0.0 | 0.4 |
| Wheat: | Finely ground flour | 0.0 | 0.0 | 0.0 | 0.0 | 2.6 | 0.0 | 0.7 | 0.0 | 0.2 |
| Robigus | Coarse ground flour | 0.0 | 0.0 | 0.0 | 0.0 | 2.5 | 0.0 | 0.5 | 0.0 | 0.2 |
| | Baked bread | 0.0 | 0.0 | 0.2 | 8.8 | 89.6 | 0.0 | 51.7 | 195.7 | 2.0 |
| | HTP-grains | 0.3 | 27.8 | 0.5 | 4.0 | 153.6 | 98.9 | 9.5 | 15.5 | 1.2 |
| Theoretical content in baked bread** | | 0.0 | 0.0 | 0.0 | 0.0 | 2.3 | 0.0 | 0.4 | 0.0 | 0.1 |
| Einkorn | Finely ground flour | 0.0 | 0.0 | 0.0 | 0.0 | 0.6 | 0.0 | 0.0 | 0.0 | 0.0 |
| | Coarse ground flour | 0.0 | 0.0 | 0.0 | 0.0 | 2.1 | 0.0 | 0.0 | 0.0 | 0.0 |
| | Baked bread | 0.0 | 0.0 | 0.0 | 0.0 | 5.0 | 0.5 | 0.8 | 2.1 | 0.2 |
| | HTP-grains | 0.0 | 7.2 | 0.2 | 1.1 | 199.9 | 62.2 | 96.8 | 112.6 | 1.8 |
| Theoretical content in baked bread** | | 0.0 | 0.0 | 0.0 | 0.0 | 1.5 | 0.0 | 0.0 | 0.0 | 0.0 |
| Spelt | Finely ground flour | 0.0 | 0.0 | 0.0 | 0.0 | 0.7 | 0.0 | 0.5 | 0.0 | 0.0 |
| | Coarse ground flour | 0.0 | 0.0 | 0.0 | 0.0 | 0.0 | 0.0 | 0.0 | 0.0 | 0.0 |
| | Baked bread | 0.0 | 5.3 | 0.1 | 1.0 | 114.4 | 35.0 | 65.1 | 63.3 | 1.3 |

| | | | | | | | | | |
|---|---|---|---|---|---|---|---|---|---|
| Theoretical content in baked bread** | 0.0 | 0.0 | 0.0 | 0.0 | 0.8 | 0.0 | 0.2 | 0.0 | 0.0 |

*mcg/g is calculated in samples with natural humidity (flours ca 35% water, HTP-grains ca 70% water); mcg is microgram
**Theoretical content in baked bread is calculated on basis of the concentrations in the ingredients and the amount of ingredient

TABLE 7
Comparison of bread baked of HTP-Kamut flour compared to bread baked of Kamut flour

| | | | | | | DiBOA- | DIM-BOA- | | DIM- | HBOA- |
|---|---|---|---|---|---|---|---|---|---|---|
| Variety/ | | BOA | MBOA | HBOA | HMBOA | glc | glc | DiBOA | BOA | glc |
| species | | mcg/g* | mcg/g* | mcg/g* | mcg/g* | mcg/g* | mcg/g* | mcg/g* | mcg/g* | mcg/g* |
| Kamut | Kamut HTP- | 15.1 | 52.2 | 2.1 | 6.0 | 7.6 | 0.7 | 3.4 | 0.0 | 0.8 |
| | grains bread | | | | | | | | | |
| | Kamut bread as | 0.0 | 0.0 | 0.1 | 0.0 | 12.1 | 0.0 | 1.1 | 0.0 | 0.9 |
| | in Example 3 | | | | | | | | | |

*mcg/g is calculated in samples with natural humidity (flours ca 35% water, HTP-grains ca 70% water); mcg are microgram
**Theoretical content in baked bread is calculated on basis of the concentrations in the ingredients and the amount of ingredient

1-21. (canceled)

22. A use of grains or disintegrated grains of benzoxazinoid-containing cereals for the manufacturing of a food product for a human or animal subject to improve the health of the subject, where the health-improving effects are selected among CNS stimulating effects, anti-cancer activity, anti-inflammatory effects, analgesic effects and antibacterial effect.

23. The use according to claim 22, where the benzoxazinoids are lactams, hydroxamic acids, benzoxazolinones, methyl-derivates, their glycosidic derivatives and other derivatives with the same basic structure.

24. The use according to claim 22, where the benzoxazinoids are one or more of DIBOA, DIBOA-Glc, DIMBOA, DIMBOA-Glc, DIM₂BOA, DIM₂BOA-Glc, BOA, MBOA, M₂BOA, HBOA, HBOA-Glc, HMBOA, HMBOA-Glc, HM₂BOA, HM2BOA-Glc, DHBOA, DHBOA-Glc, 4-O-Me-DIBOA, 4-O-Me-DIBOA-Glc, HDMBOA, HDMBOA-Glc as well as chlorinated and hydroxylated derivatives, isomers, steroisomers and enantiomers thereof.

25. The use according to claim 22, where the grains are selected among the group consisting of grains of rye, maize, wheat, kamut, einkorn, spelt, wild barley, rice, oat, sorghum, millet, teff, buckwheat, amaranth, quinoa, durum and triticale.

26. The use according to claim 22, where the grains are grains of rye.

27. The use according to claim 22, where the grains are pre-treated by being contacted with water one or more times during a period of at least two days.

28. The use according to claim 22, where the manufacture of the food product comprises heat treatment of the cereal grain-containing food products or beverages.

29. The use according to claim 28, where the food product is baked bread.

30. The use according to claim 22, where the CNS stimulating effects are selected among appetite suppression, enhancement of mood, improved sexual function, relieve of fibromyalgia and relieve of sleep apnea disorders.

31. Grains or disintegrated grains of benzoxazinoid-containing cereals for use in a human or animal subject to improve the health of the subject, where the health-improving effects are selected among CNS stimulating effects, anti-cancer activity, anti-inflammatory effects, analgesic effects and antibacterial effect.

32. The grains or disintegrated grains according to claim 31, where the grains are selected among the group consisting of grains of rye, maize, wheat, kamut, einkorn, spelt, wild barley, rice, oat, sorghum, millet, teff, buckwheat, amaranth, quinoa, durum and triticale.

33. The grains or disintegrated grains according to claim 31, where the grains are grains of rye.

34. The grains or disintegrated grains according to claim 31, where the grains are pre-treated by being contacted with water one or more times during a period of at least two days.

35. The grains or disintegrated grains according to claim 31, where the manufacture of the food product comprises heat treatment of the cereal grain-containing food products or beverages.

36. The grains or disintegrated grains according to claim 35, where the food product is baked bread.

37. The grains or disintegrated grains according to claim 31, where the CNS stimulating effects are selected among appetite suppression, enhancement of mood, improved sexual function, relieve of fibromyalgia and relieve of sleep apnea disorders.

38. A method of producing an increased content of benzoxazinoids in grains of benzoxazinoid-containing cereals, the method comprising the steps of a) contacting the grains with water, and b) resting or drying the grains, and c) optionally repeating the steps a) and b), during a period of at least two days.

39. The method according to claim 38 further comprising a step d) of heat treating the grains or disintegrated grains at a temperature of between 60° C. and 300° C.

40. A food product comprising grains or disintegrated grains of benzoxazinoid-containing cereals characterised in that the cereal grains have been pre-treated by being contacted with water and dried one or more times during a period of at least two days.
